(19) United States
(12) Patent Application Publication
(10) Pub. No.: US 2023/0088271 A1
(43) Pub. Date: Mar. 23, 2023

(54) Systems and Methods for Determining Spatial Accumulation of Signaling Molecules Within Tissue Samples

(71) Applicant: The Board of Trustees of the Leland Stanford Junior University, Stanford, CA (US)

(72) Inventors: Helen M. Blau, Menlo Park, CA (US); Yu Xin Wang, Stanford, CA (US); Colin A. Holbrook, Palo Alto, CA (US)

(73) Assignee: The Board of Trustees of the Leland Stanford Junior University, Stanford, CA (US)

(21) Appl. No.: 17/905,250

(22) PCT Filed: Mar. 1, 2021
(86) PCT No.: PCT/US21/20334

§ 371 Date: Aug. 29, 2022

Related U.S. Application Data
(60) Provisional application No. 62/983,476, filed on Feb. 28, 2020.

(51) Int. Cl. G16B 5/20; G16B 25/10

(52) U.S. Cl. CPC G16B 5/20; G16B 25/10

(57) ABSTRACT

The present disclosure describes systems and methods for determining spatial accumulation of signaling molecules within tissue samples. Embodiments of the present disclosure are directed to integrating spatial and expression data for cells in a tissue. Embodiments further describe identifying cell linkages and rendering transcriptome profiles to spatial coordinates. Some embodiments further convolve diffusion information for various ligands and measure effective concentrations within cell areas. Using this information, embodiments are able to predict cell-cell signaling information.

### CROSS-REFERENCE TO RELATED APPLICATIONS

This application claims the benefit of U.S. Provisional Patent Application No. 62/983,476 entitled “Systems and Methods for Determining Spatial Accumulation of Signaling Molecules within Tissue Samples” filed Feb. 28, 2020, which is incorporated by reference herein in its entirety.

### FIELD OF THE INVENTION

The present invention relates generally to determining the spatial distribution of signaling molecules in tissue. More particularly, the invention relates computational methods for spatially modeling signaling molecules and cells types to identify cell-cell signaling pathways.

### BACKGROUND OF THE INVENTION

Cellular signaling is critical to understand disease mechanisms and predictions of cell-cell signaling (e.g. vascular-endothelial growth factor, insulin-like growth factor-1, Wnt, and Notch signaling) and is commonly used to identify novel therapeutics. This has led to the use of transcriptomics approaches to determine the expression levels of ligands, growth factors, and other signaling molecules. However, these analyses are done without consideration of spatial interactions between cell types within a tissue. However, this commonly results in false positive in predicted effectors of biological processes and hinders the discovery of disease mechanisms and drug targets. Moreover, the spatial localization of cellular signaling molecules are difficult to determine by traditional approaches (immunohistochemistry and mass spectrometry), thus an unbiased discovery tool is necessary to bridge this gap.

### SUMMARY OF THE INVENTION

Systems and methods for determining spatial accumulation of signaling molecules within tissue samples in accordance with embodiments of the invention are disclosed.

In one embodiment, a method to predict cellular signaling pathways including obtaining spatial data and expression data, wherein the spatial data identifies cell type and cell position within a sample based on imaging of the tissue, and where the expression data identifies genes being expressed in individual cells, aligning and integrating the spatial and expression data to identify cell linkages between cell type and expression data, rendering spatial transcriptomics data, where the spatial transcriptomics data comprises a transcriptome profile for a cell in the sample in spatial coordinates based on the spatial data and cell linkages, generating a ligand diffusion map based on spatial data and ligand diffusion information, measuring effective ligand concentration at cellular positions based on the ligand diffusion map, and predicting cell-cell signaling in the sample based on the concentration of a ligand at a cellular position within the sample.

In a further embodiment, the sample is a tumor from an individual.

In another embodiment, the method further includes identifying a treatment to block or modulate the cell-cell signaling within the tumor, and treating an individual with the treatment to block or modulate the cell-cell signaling.

In a still further embodiment, the expression data includes single cell RNA sequencing data.

In still another embodiment, the expression data includes bulk RNA sequencing data.

In a yet further embodiment, the spatial data is obtained from multiparametric tissue imaging.

In yet another embodiment, the spatial data includes annotated cell type information.

In a further embodiment again, the spatial data includes single cell protein expression data.

In another embodiment again, the expression data includes single cell RNA sequencing (scRNA-Seq) data.

In a further additional embodiment, aligning and integrating the spatial and expression data includes linking a subset of scRNA-Seq data to annotated cell type to create cell lineages, linking protein makers to corresponding genes based on the single cell protein expression data and the expression data, and creating a distance matrix for cells across the scRNA-Seq data and the spatial data.

In another additional embodiment, generating a ligand diffusion map includes obtaining ligand information from a database, where the ligand information includes the ligand diffusion information, calculating a diffusion kernel for a ligand based on the ligand diffusion information, and convolving the spatial transcriptomics data with the diffusion kernel.

In a still yet further embodiment, the ligand information further includes. at least one of the following: signaling modality, gene name, molecular property, and diffusion constant.

In still yet another embodiment, measuring effective ligand concentration includes convolving the diffusion map with cell radius assess an effective ligand concentration over a cell area and outputting ligand concentration for a cell.

In a still further embodiment again, a non-transitory machine-readable media containing processor instructions, where execution of the instructions causes a processor to predict cellular signaling pathways including obtaining spatial data and expression data, wherein the spatial data identifies cell type and cell position within a sample based on imaging of the tissue, and where the expression data identifies genes being expressed in individual cells, aligning and integrating the spatial and expression data to identify cell linkages between cell type and expression data, rendering spatial transcriptomics data, where the spatial transcriptomics data comprises a transcriptome profile for a cell in the sample in spatial coordinates based on the spatial data and cell linkages, generating a ligand diffusion map based on spatial data and ligand diffusion information, measuring effective ligand concentration at cellular positions based on the ligand diffusion map, and predicting cell-cell signaling in the sample based on the concentration of a ligand at a cellular position within the sample.

In still another embodiment again, the sample is a tumor from an individual.

In a still further additional embodiment, the instructions further include identifying a treatment to block or modulate the cell-cell signaling within the tumor.

In still another additional embodiment, the expression data includes single cell RNA sequencing data.

In a yet further embodiment again, the expression data includes bulk RNA sequencing data.

In yet another embodiment again, the spatial data is obtained from multiparametric tissue imaging.

In a yet further additional embodiment, the spatial data includes annotated cell type information.

In yet another additional embodiment, the spatial data includes single cell protein expression data.

In a further additional embodiment again, the expression data includes single cell RNA sequencing (scRNA-Seq) data.

In another additional embodiment again, the aligning and integrating the spatial and expression data includes linking subset of scRNA-Seq data to annotated cell type to create cell lineages, linking protein makers to corresponding genes based on the single cell protein expression data and the expression data, and creating a distance matrix for cells across the scRNA-Seq data and the spatial data.

In a still yet further embodiment again, generating a ligand diffusion map includes obtaining ligand information from a database, wherein the ligand information includes the ligand diffusion information, calculating a diffusion kernel for a ligand based on the ligand diffusion information, and convolving the spatial transcriptomics data with the diffusion kernel.

In still yet another embodiment again, the ligand information further includes at least one of the following: signaling modality, gene name, molecular property, and diffusion constant.

In a still yet further additional embodiment, measuring effective ligand concentration includes convolving the diffusion map with cell radius assess an effective ligand concentration over a cell area and outputting ligand concentration for a cell.

In still yet another additional embodiment, a system for predicting cellular signaling pathways includes a processor and a memory readable by the processor, where the memory contains instructions that when read by the processor direct the processor to obtain spatial data and expression data, wherein the spatial data identifies cell type and cell position within a sample based on imaging of the tissue, and wherein the expression data identifies genes being expressed in individual cells, align and integrating the spatial and expression data to identify cell linkages between cell type and expression data, render spatial transcriptomics data, wherein the spatial transcriptomics data comprises a transcriptome profile for a cell in the sample in spatial coordinates based on the spatial data and cell linkages, generate a ligand diffusion map based on spatial data and ligand diffusion information, measure effective ligand concentration at cellular positions based on the ligand diffusion map, and predict cell-cell signaling in the sample based on the concentration of a ligand at a cellular position within the sample.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

These and other features and advantages of the present invention will be better understood by reference to the following detailed description when considered in conjunction with the accompanying drawings where:

FIG. 1A illustrates a method of determining spatial accumulation of signaling molecules within tissue samples in accordance with various embodiments.

FIG. 1B illustrates exemplary spatial transcriptome profiles in accordance with various embodiments.

FIG. 2A illustrates a method to align and integrate spatial and expression data in accordance with various embodiments.

FIG. 2B illustrates an exemplary graphical model for generating spatial transcriptome profile in accordance with various embodiments.

FIG. 3 illustrates a method diffusion and measurement in accordance with various embodiments.

FIG. 4 illustrates a method to identify cell-cell contact signaling in accordance with various embodiments.

FIG. 5 illustrates an exemplary schematic overview for data integration, spatial transcriptome mapping, and growth factor diffusion modelling.

FIGS. 6A-6B illustrate exemplary major cell types in regenerating skeletal muscle in accordance with various embodiments. Specifically, FIG. 6A illustrates an unsupervised clustering results for single cell RNA-sequencing of mononuclear cells isolated from a time course of muscle regeneration, and FIG. 6B illustrates a skeletal muscle CODEX antibody panel.

FIG. 7 illustrates exemplary CODEX images of regenerating skeletal muscle showing accurate detection of 40 antibodies and simultaneous visualization of myogenic and immune cell types in accordance with various embodiments.

FIG. 8 illustrates exemplary spatial maps of 27 cell types in uninjured and regenerating TA muscles (days 3 and 6 after injury) of wild type mice in accordance with various embodiments.

FIG. 9 illustrates exemplary predictions of interleukins with distinct spatial localization and their quantified “influence” on myogenic subsets in accordance with various embodiments.

FIGS. 10A-10D illustrate exemplary data regarding colon cancer prognosis and modeling in accordance with various embodiments.

### DETAILED DESCRIPTION

The embodiments of the invention described herein are not intended to be exhaustive or to limit the invention to precise forms disclosed. Rather, the embodiments selected for description have been chosen to enable one skilled in the art to practice the invention.

Turning now to the drawings, embodiments of the invention align multiplexed imaging data to resolve the positioning information of multiple cell types and single cell transcriptomics data, using either a semi-supervised alignment algorithm or a pre-trained neural network. Many embodiments project the expression of mRNAs within the tissue at a higher resolution than current spatial transcriptomics approaches. Additional embodiments are capable using databases of signaling proteins and molecules, along with their estimated diffusion constants to predict the accumulation of these signaling molecules within the tissue based on expression level and positioning of source cells. By using this prediction capability, certain embodiments are capable of calculating the potential effect of ligands on each cell type of interest and allow more accurate predictions of cell-cell signaling. By improvements in predicting cell-cell signaling, additional embodiments serve as potent drug discovery tools.

A number of embodiments use multiplexed imaging methods to capture cell types in a tissue specimen and single cell spatial information. Moreover, numerous embodiments integrate spatial data with single cell gene expression profiling by scRNA-Seq to predict the localization and accumulation of ligands within a tissue. This approach in many embodiments is applicable in various disease settings, whereby these embodiments can identify ligands and signaling pathways that affect various biological processes of interest (e.g., muscle stem cell expansion, myogenic differentiation, immune cell infiltration, fibrosis, neuromuscular interactions, cancers, drug responses, etc.). Further embodiments can leverage this information to generate better treatments and treatment protocols for use in medicine and medical research.

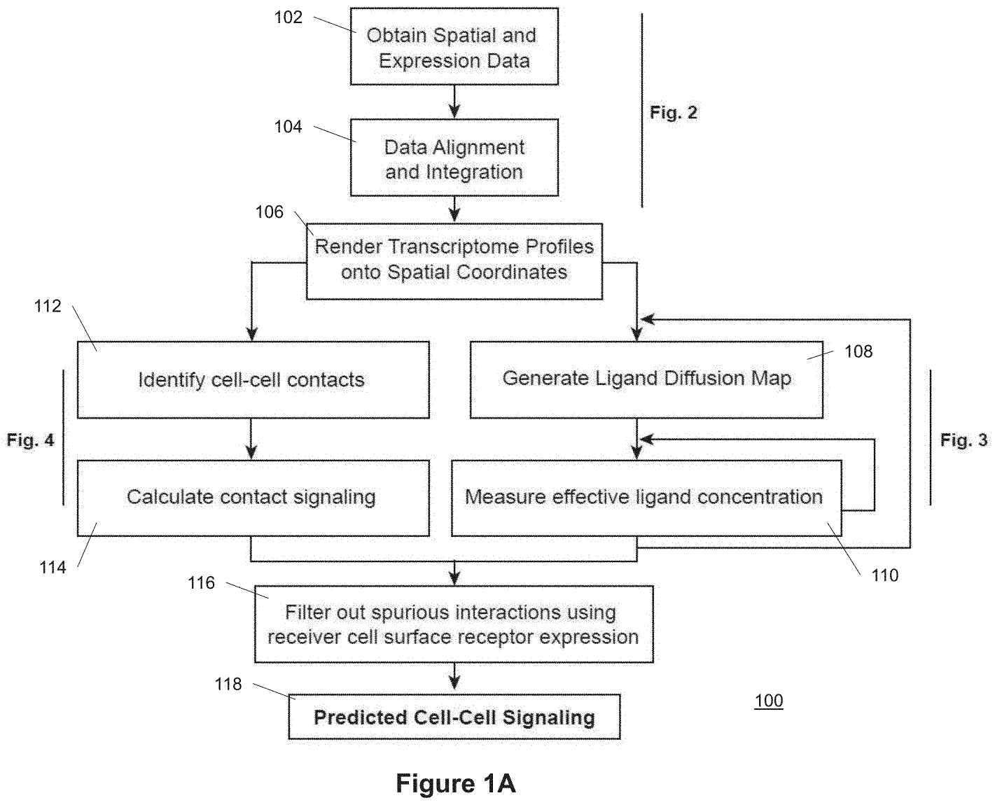

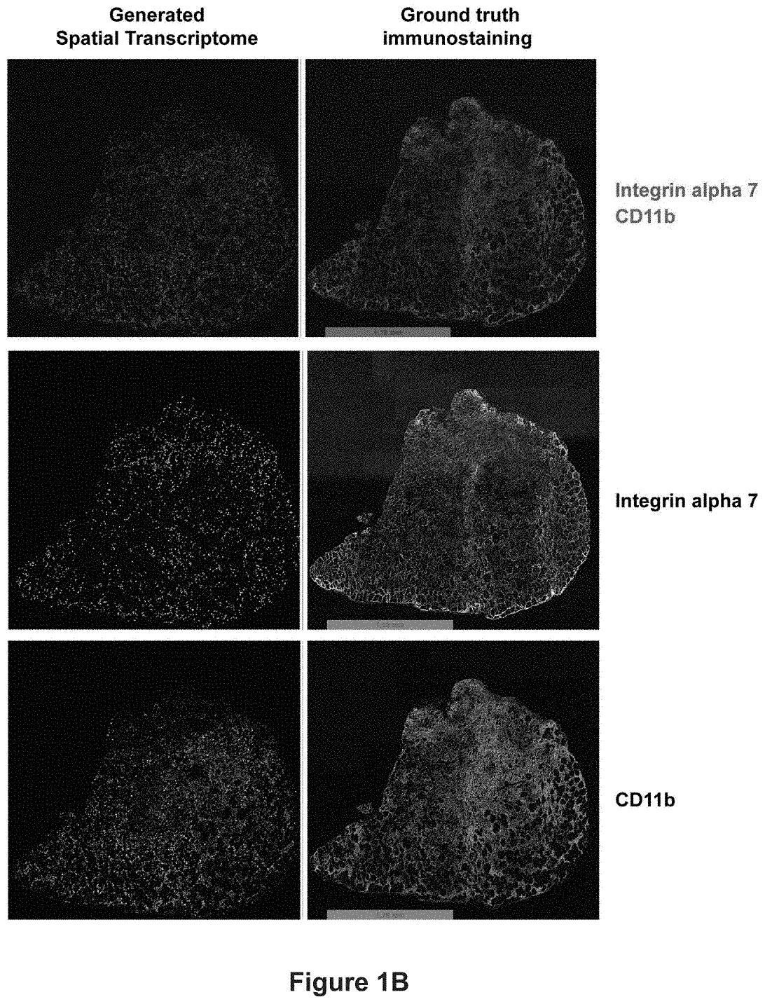

Turning to FIG. 1A, a method 100 to predict cell-cell signaling in accordance with many embodiments is illustrated. In particular, many embodiments obtain spatial data and expression data (102). In a number of embodiments, the spatial data is obtained via a system for spatial profiling, such as CODEX (CO-Detection by indEXing), which identifies cellular positions within tissue based on imaging of particular cellular antigens. (See e.g., Goltsev, Y. et al. Deep Profiling of Mouse Splenic Architecture with CODEX Multiplexed Imaging. Cell 174, 968-981.e15 (2018); the disclosure of which is hereby incorporated by reference in its entirety.)

In many embodiments, a multi-antibody detection pool is capable of identifying spatial distributions of multiple cell types. Certain embodiments perform microscopy assays to image and distinguish cell types to generate the spatial profiling data, such as described in PCT/US2021/020299, entitled “Systems and Methods for Composing High Dimensional Microscopy Images in Subpixel Resolution” (the disclosure of which is hereby incorporated by reference in its entirety), which demonstrate methods of obtaining spatial profiling data from tissue by composing high dimensional fluorescence images with subpixel resolution. Certain embodiments utilize a CODEX panel, including a panel of 40 antibodies to simultaneously capture spatial distributions of 25 cell types within a tissue. Some embodiments apply this methodology to model organisms (e.g., mice, rat, rabbit, plant), while additional embodiments apply this methodology to humans. In human tissue, many embodiments obtain tissue from a tumor or other neoplasm. In certain embodiments, the tissue sample is obtained as a biopsy or other tissue collection method. Upon collection of such tissue, the tissue is profiled, such as through CODEX or another tissue profiling system to identify spatial data and identification data for cells within the tissue.

In various embodiments, the expression data is obtained from RNA profiling, such as RNA sequencing that identifies which genes are being expressed in a particular cell and/or tissue, including single cell RNA sequencing (scRNA-seq). In some embodiment, additional data captured during scRNA-seq can also include simultaneous measurements of protein expression using antibodies such as cellular indexing of transcriptomes and epitopes by sequencing (CITE-seq; see e.g., Stoeckius et. al Simultaneous epitope and transcriptome measurement in single cells. Nature Methods 14, 865-868 (2017); the disclosure of which is hereby incorporated by reference in its entirety.) Some embodiments obtain the transcription data from microarray, bead array, or any other method for obtaining expression data. Some embodiments obtain transcription from a database of expression data with single cell resolution, while additional embodiments obtain expression data via cell sorting (e.g., via flow cytometry) and de novo sequencing of cells from the sample.

At 104 of many embodiments, the data obtained in 102 is aligned and integrated in order to identify cellular linkages. Once cellular linkages are identified, many embodiments render transcriptome profiles into spatial coordinates at 106. In embodiments using CODEX, the transcriptome profiles are input in line with the CODEX coordinates. Specific processes to perform steps 102-106 are discussed in greater detail below. FIG. 1B illustrates exemplary spatial transcriptomics data rendered in accordance with various embodiments as compared to ground truth immunostained imaging. Specifically, the spatial transcriptome illustrates the location of integrin alpha 7 and CD11b as compared to in situ imaging showing the ground truth location of integrin alpha 7 and CD11b. The similar patterns of projected expression indicate that embodiments accurately render transcriptomic data relative to actual expression within tissue.

Returning to FIG. 1A, once the transcriptome profiles are spatially rendered (e.g., at 106), many embodiments generate a ligand diffusion map by convolving the spatial information with ligand diffusion information (108). Ligand diffusion information is derived from information, such as gene names, signaling modality, molecular properties, and/or diffusion constants for ligands identified by spatial and/or expression data. Certain embodiments experimentally determine ligand diffusion information, while other embodiments obtain ligand diffusion information from databases.

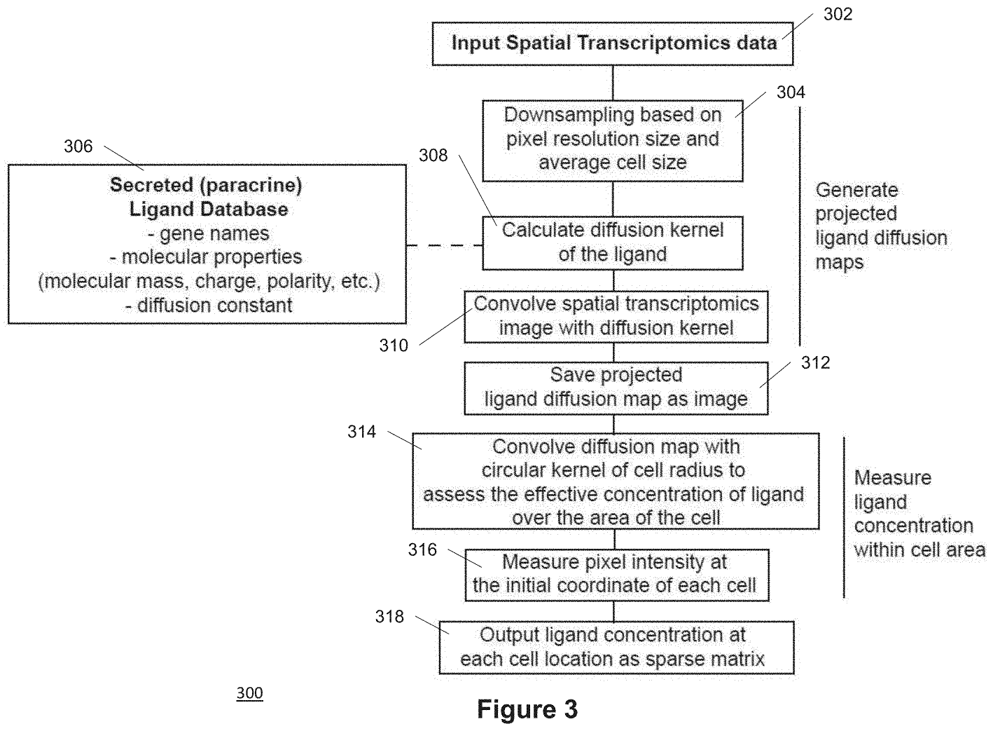

At 110, many embodiments measure effective ligand concentration within a cellular area. A number of embodiments iteratively perform 110 for each cell in a particular region, tissue, and/or organ. Similarly, many embodiments iteratively perform 108-110 for each ligand and each cell in a particular region, tissue, and/or organ. Specific processes to perform 108-110 are discussed in greater detail in reference to FIG. 3.

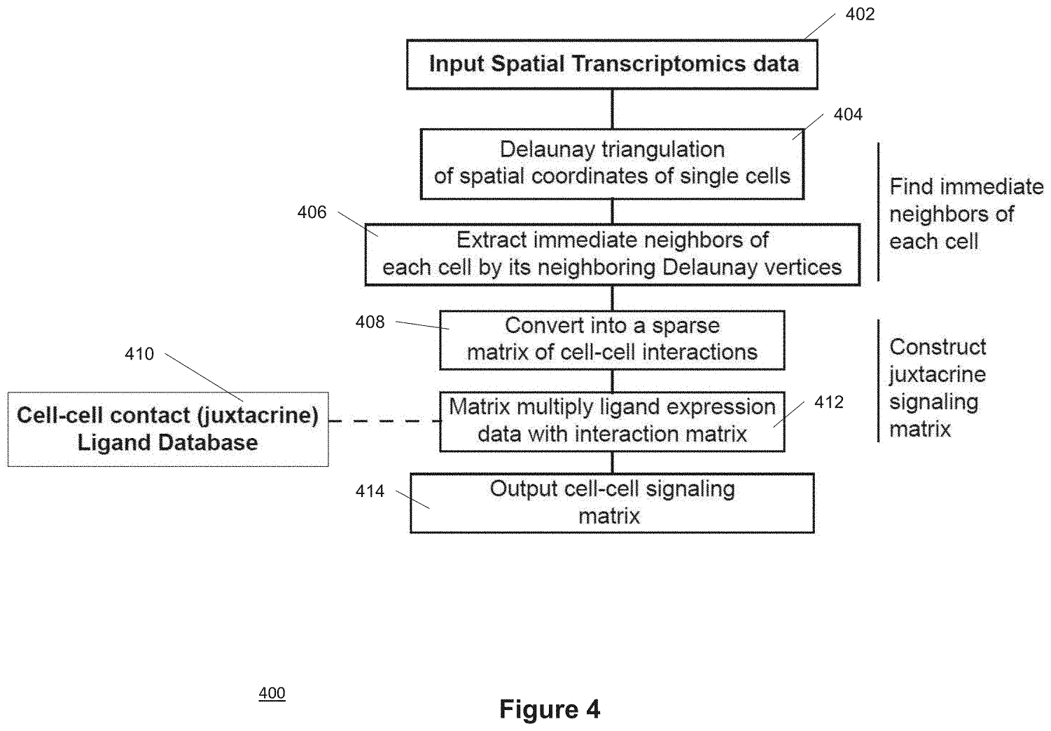

Further embodiments identify cell-cell signaling (e.g., signaling between neighboring cells) by identifying cell-cell contacts (112) and calculating contact signaling between such contacts (114). Such embodiments determine cell-cell signaling, when ligands do not diffuse through tissue or are effects of receptor-ligand interactions between neighboring cells. Specific processes to perform 112-114 are discussed in greater detail in reference to FIG. 4.

At 116, various embodiments filter out spurious interactions based on surface receptor expression. For example, if a cell does not express a surface receptor for ligands in the vicinity of a cell or capable of receiving signals from neighboring cells, such spurious interactions can be filtered out at 116.

At 118, many embodiments predict cell-cell signaling based on the effective ligand concentration of each ligand within each cellular area in the particular region, tissue, and/or organ.

Upon identifying cell-cell signaling, various embodiments identify drugs or treatments capable of treating or a disorder. For example, in embodiments that identify cell-cell signaling in tumor or other diseased tissue, these embodiments can identify whether blocking or modulating certain signaling pathways may treat, cure, or alleviate the disease or disorder. If signaling pathways can be used for treatment (e.g. stimulate T-cell response against tumor cells that secrete immune evasion molecules), certain embodiments treat the individual with a drug capable of blocking or modulating the signaling pathway. In certain embodiments, the drug blocks a surface receptor on a cell, while in additional embodiments, the drug binds a ligand directly. Drugs in accordance with various embodiments include small molecules and biologics, including antibodies (e.g., monoclonal antibodies) and traps. Certain embodiments treat an individual with anti-VEGF (anti-vascular endothelial growth factor) treatments, such as ranibizumab, bevacizumab, aflibercept, lapatinib, sunitinib, sorafenib, axitinib, and pazopanib. Additionally, some embodiments provide anti-CGRP (anti-calcitonin gene-like receptor protein) treatments, including erenumab, eptinezumab, fremanezumab, galcanezumab, and atogepant. Further embodiments treat an individual for anti-cytokine therapies, such as anti-IL6, including tocilizumab, siltuximab, sarilumab, olokizumab, elsilimomab, sirukumab, levilimab, and clazakizumab. It should be noted that the embodiments listing anti-VEGF, anti-CGRP, and anti-IL6 treatments are merely illustrative and embodiments are capable of identifying numerous signaling pathways that may affect disease and/or disease progression. One of skill in the art would understand drugs that affect such pathways.

### Data Alignment and Integration

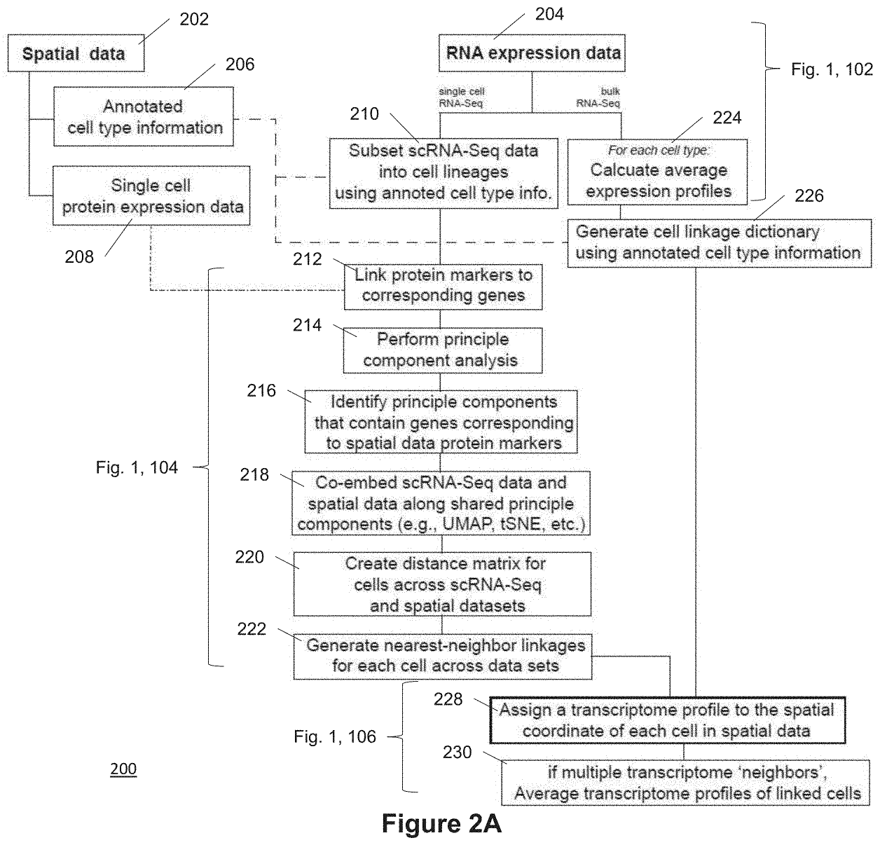

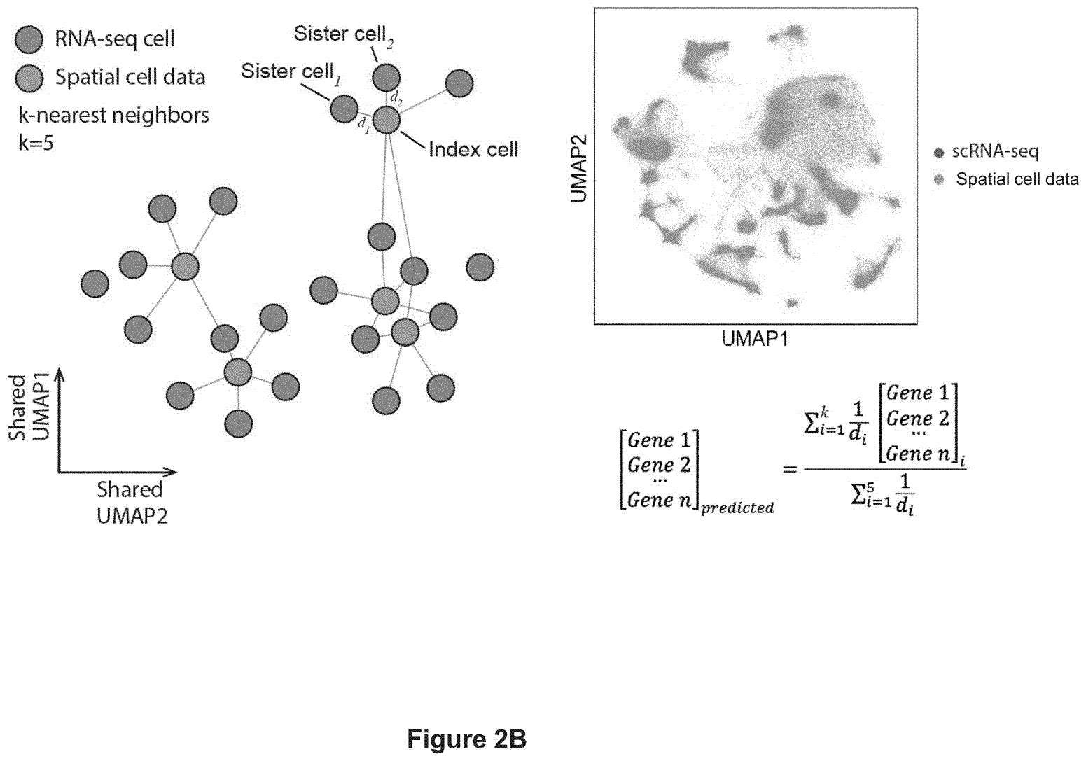

Turning to FIG. 2A, method 200 illustrates additional detail used in various embodiments to align and integrate spatial data and expression data (e.g., FIG. 1A, 104-106). In many embodiments, method 200 begins with spatial data 202 and RNA expression data 204, such as obtained in method 100 (FIG. 1A, 102). As noted in reference to FIG. 1A, some embodiments obtain spatial data as data from a CODEX panel. As mentioned previously, some embodiments generate spatial data through improved methods to compose high dimensional microscopy images with subpixel resolution. In certain embodiments, the spatial information 202 includes annotated cell type information 206 and single cell protein expression data 208 as determined by labeling of the tissues in the spatial data. For example, embodiments using CODEX, antibodies and other probes can provide information or annotation about the cell types present in the tissue based on the specific probes or and/or antibodies that hybridize to specific cells. Various embodiments utilize one or more machine learning models to annotate cell type information from the obtained spatial data. For example, if spatial data identifies cells at specific locations and types of markers, the one or more machine learning models can annotate the cell type information based on the markers or identifying information present within the spatial data.

Additionally, expression data can include single cell RNA sequencing (scRNA-Seq) data as well as bulk RNA sequencing data (e.g., RNA data generated from RNA sequenced from a collection of cells of individual cell-types). Single cell RNA sequencing data can also include simultaneous capture of protein using antibodies (CITE-seq; Stoeckius et. al Simultaneous epitope and transcriptome measurement in single cells. Nature Methods 14, 865-868 (2017); the disclosure of which is hereby incorporated by reference in its entirety), which can be used to provide direct correlation with single cell proteins measurements in the spatial data such as fluorescence intensity from CODEX data.

In some embodiments, a subset of scRNA-Seq data is put into cell lineages 210, based on annotated cell type information 206 (e.g., from the spatial data), combining the cell lineages 210 with the single cell protein expression data 208 (e.g., from spatial data) allows the linking of protein markers to corresponding genes 212, in various embodiments. A principle component analysis (PCA) 214 can be used, which allows the identification of principle components 216 that contain genes corresponding to the spatial data (e.g., CODEX protein markers). In many embodiments, the combination of scRNA-Seq data, spatial data, and the shared principle components is co-embedded 218, which allows the creation 220 of a distance matrix for cells across the scRNA-Seq data and spatial data. Additionally, certain embodiments generate nearest-neighbor linkages 222 for each cell across the scRNA-Seq data and spatial data based on the distance matrix.

With the bulk RNA sequencing data, the average expression profile 224 for each cell type is calculated in many embodiments, which can be combined with annotated cell type information 206 (e.g., from spatial data) to generate a cell linkage dictionary 226. In many embodiments, this cell linkage dictionary is combined with the nearest-neighbor linkages 222 to assign a transcriptome profile or a distance-weighted average transcriptome profile of its nearest neighbors to the spatial coordinate of each cell 228 in the spatial data (e.g., CODEX data). In some embodiments, if multiple neighbors are identified in the transcriptome profile, transcriptome profiles of the linked cells are averaged 230. FIG. 2B graphically illustrates an exemplary process for generating spatial transcriptomics data in accordance with method 200, by identifying index cells with sister cells and mapping scRNA-Seq data to spatial cell data.

Further embodiments are able to provide expression data based on the protein expression, such as obtained by probes, antibodies, biomarkers, etc. In various embodiments, the protein expression data is obtained from the spatial data, such as by intensity or brightness of the probes.

### Diffusion and Measurement

Turning to FIG. 3, method 300 illustrates additional details used in several embodiments to generate ligand diffusion maps and measure ligand concentration within a cell area. In method 300, a number of embodiments obtain spatial transcriptomics data 302. In many embodiments, spatial transcriptomics data 302 identifies gene expression based on its three-dimensional position within a tissue. In certain embodiments, the spatial transcriptomics data is obtained from 106, method 100 (see FIG. 1A) and/or the output of method 200 (see FIG. 2A).

To generate the projected ligand diffusion maps, certain embodiments downsample the spatial transcriptomics data 304 based on pixel resolution and average cell size. Pixel resolution is identifiable from the spatial data that has been integrated into the spatial transcriptomics data. Additional embodiments obtain secreted (paracrine) ligand information (306), such as from a ligand database that includes information for ligands, including (but not limited to) gene names, molecular properties (e.g., molecular mass, charge, polarity), diffusion constants, and any other relevant information for identifiable ligands. Using ligand information, certain embodiments calculate a diffusion kernel 306 for each ligand. In further embodiments, the diffusion kernel is then convolved with the spatial transcriptomics image 310 to generate a projected ligand diffusion map. This projected ligand diffusion map is saved 312 as an image in many embodiments.

To measure ligand concentration within a cell area, many embodiments convolve the diffusion map with a circular kernel of cell radius 314 to assess the effective concentration of ligand over the area of the cell. Additional embodiments measure pixel intensity in the diffusion map 316 at the initial coordinate of each cell, and further embodiments output ligand concentrations at each cell location 318. Many embodiments output the ligand concentrations as a sparse matrix. Some embodiments store the matrix in a suitable file form, such as comma separated, tab delimited, or any other suitable form that displays a concentration of one or more ligands in the area of a cell. By producing both a diffusion map and identifying the relative concentration in the area of a cell, many embodiments provide a clearer understanding of which signaling interactions are actually occurring within a tissue.

### Cell Contact Signaling

Turning to FIG. 4, method 400 illustrates additional details used in several embodiments to identify cell-cell contact signaling. In method 400, a number of embodiments obtain spatial transcriptomics data 402. In many embodiments, spatial transcriptomics data 302 identifies gene expression based on its three-dimensional position within a tissue. In certain embodiments, the spatial transcriptomics data is obtained from 106, method 100 (see FIG. 1A) and/or the output of method 200 (see FIG. 2A).

To find immediate neighbors of each cell, various embodiments perform Delaunay triangulation of spatial coordinates of each cell at 404. By identifying Delaunay coordinates for each cell, further embodiments extract immediate neighbors of each cell by neighboring Delaunay vertices at 406.

Further embodiments construct a juxtacrine signaling matrix to identify signaling from neighboring cells. To construct such matrices, certain embodiments convert immediate neighbors into a sparse matrix of cell-cell interactions at 408. Further embodiments obtain cell-cell contact (juxtacrine) ligand information 410, such as from a database that includes information for ligands, including (but not limited to) gene names, molecular properties (e.g., molecular mass, charge, polarity), expression information, and any other relevant information for identifiable ligands. Using ligand information, certain embodiments matrix multiply ligand expression with an interaction matrix (e.g., from 408). Further embodiments output ligand concentrations at each cell location 318. Many embodiments output a cell-cell signaling matrix at 414. Some embodiments store the matrix in a suitable file form, such as comma separated, tab delimited, or any other suitable form.

In certain embodiments, a non-transitory machine-readable media is provided containing processor instructions, where execution of the instructions causes a processor to execute a method similar to any of the methods described above with reference to FIGS. 1A, 2A, & 3-4. Further, image processing systems in accordance with many embodiments of the invention include a processor and memory, where the memory contains software that configures the processor to perform a process similar to one of the processes described above with reference to FIGS. 1A, 2A & 3-4.

### Exemplary Embodiments

Although the following embodiments provide details on certain embodiments of the inventions, it should be understood that these are only exemplary in nature, and are not intended to limit the scope of the invention.

### Example 1: Predicting Inter-Cellular Signaling in Skeletal Muscle Regeneration

Background: The microenvironment, or cell niche, is a critical determinant of cell behavior. Spatial regulation between cell types occurs through localized communication via secreted paracrine factors and contact-dependent juxtacrine signals. In the context of injury, the release of factors from damaged cells elicit a pro-inflammatory response that recruits multiplicity of cell types to the injury site. Efforts to understand inter-cellular regulation during regeneration have revealed heterogeneous cell populations and candidate signaling pathways that are critical for efficacious regeneration. However, an understanding of the spatial relationships between cells, in the context of the tissue architecture, has been limited by technological barriers in microscopy and by the requirement for tissue dissociation in single-cell transcriptomics approaches. It is still unknown how the multiplicity of cell types spatially interact with other cells in the context of the tissue microenvironment. Thus, tissue cytometry, the mapping of single cells within tissues, is necessary to gain novel insights into the spatiotemporal dynamics of muscle regeneration and disease. This project capitalizes on two cutting edge single-cell technologies, (1) multiparametric tissue imaging (also known as CODEX, CO-Detection by indEXing) to map cellular dynamics in situ and (2) integration with single cell transcriptomics to systematically identifies spatial regulators of tissue regeneration.

There are only a few examples of cellular interactions in the literature, due to the limitations of tools available to biologists. Furthermore, almost nothing is known regarding cell-cell contact mediated signaling from the lack of ability to generate spatial information across multiple cell types. Thus, a systematic approach to study cellular interactions that occur during muscle regeneration and disease has significant potential for identifying novel pathways critical to coordinate the repair process. This embodiment overcomes these limitations by developing novel methods to systematically study cellular interactions in situ and predict spatially localized signals that can affect cellular processes and tissue regeneration.

As localized signals provide instructions for cells to organize into a functioning tissue, it is expected that cell-cell signaling within regenerating tissues or in disease can be modeled based on spatial positions of each cell type within the tissue and their expression of various ligands and receptors. Further, since multiple cell types accumulate regionally within the tissue, signaling molecules do not exclusively come from one cell type to signal to another. Thus, previous methods of predicting cell-cell signaling based on transcriptomics could not accurately account for the spatial accumulation of ligands or the proximity of recipient cells to the signal source. To address this need, this example uses an optimized multiplexed imaging method to capture 25 cell types in muscle tissue and single cell spatial information. Moreover, this embodiments integrates spatial data with single cell gene expression profiling by scRNA-Seq to predict the localization and accumulation of ˜2000 ligands within a tissue. This approach will be applicable in various disease settings, whereby ligands and signaling pathways can be identified that highly affect various biological processes of interest (e.g., muscle stem cell expansion, myogenic differentiation, immune cell infiltration, fibrosis, neuromuscular interactions).

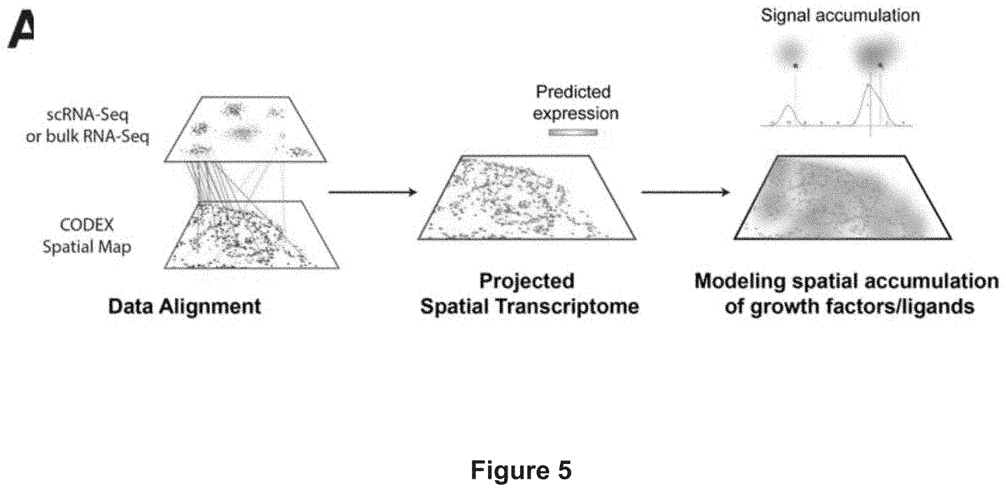

Methods: Linkages were created across these datasets by finding the nearest neighbor, or the “sister cell”, of each cell in the combination of scRNA-Seq and CODEX datasets in high dimensional space (FIG. 5; left). This linkage is non-trivial and requires a complex data alignment algorithm. The linkage labels each cell captured by CODEX with the transcriptome profile of its “sister cell” captured by scRNA-Seq, thus generating an imputed spatial transcriptome (FIG. 5; middle). Further, using a curated database of ligands, growth factors, and adhesion proteins with their calculated diffusion constants and signaling method (paracrine or juxtacrine), the spatial distribution of each signaling molecules can be modeled based on expression levels (FIG. 5; right). This accounts for the localized accumulation of signaling molecules within the tissue and estimates the effective concentration of all˜2000 signaling molecules experienced by any given cell. This potent combination enables the identification of signals that are spatially correlated with cellular functions (e.g. myogenic differentiation, cell proliferation, etc.).

To map spatial dynamics of multiple cell types in situ, CODEX multiplexed immunofluorescence imaging was utilized. CODEX enables the visualization of up to 60 proteins (cell surface markers, cell cycle proteins, transcription factors, ECM proteins) on single frozen tissue section.

In short, antibodies are labelled with unique DNA barcodes to circumvent spectral overlap and allow for reversible visualization using fluorophore-conjugated complementary DNA probes. The CODEX imaging process occurs in cycles: (i) probes are flowed over the antibody-labelled tissue sections 3 at a time using a pre-programmed microfluidics device (Akoya Biosciences); (ii) once stained, images are captured in 3D on an automated microscope at 20× magnification; (iii) using chemistries that modify the melting temperature of DNA, the probes can be denatured and washed off, allowing for subsequent staining with other probes; (iv) this is repeated until all antibodies are imaged.

Overall, 80,000-100,000 images are captured per tissue (captured in frames tiled along width and height corresponding to the X and Y axis; at different focal depth along the Z-axis; across 40+ antibodies, DNA stains, and autofluorescence signal imaged in cycles at different times). The images are processed through a comprehensive image processing pipeline. This algorithm uses Fourier transformation to find correlations between images in 3D, and uses force convergence to refine drift compensation offsets of images within a known grid. CRISP is also able to correct for the tilt or skew of the tissue when it was imaged by finding best focus slices of a 3D image stack, as to generate a flattened version of the tissue with a single plane of the most in focus images. Wide field images are deconvolved using the Richardson-Lucy deconvolution. The tiled images are corrected for chromatic aberration, registered using information across all channels and cycles, translated in X and Y, and placed into a stitched mosaic of tiles.

This system allows the generation of highly accurate alignment of high dimensional images, thus better colocalization of signals across channels that can be viewed across imaging cycles. Moreover, the image products allow for better segmentation, background subtraction, and quantification of marker intensity.

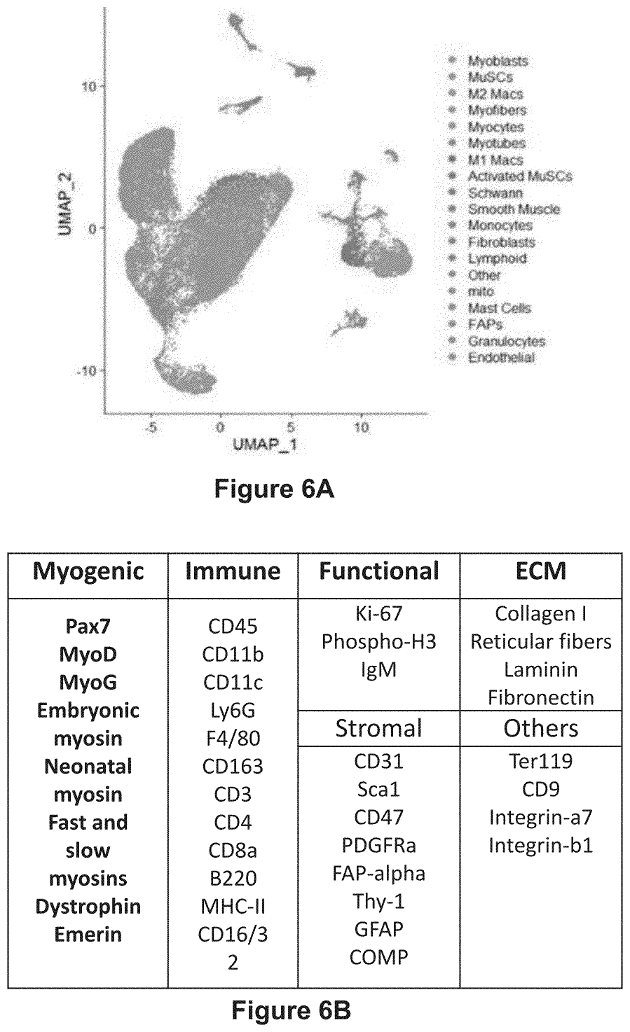

Results: Early estimates of homeostatic skeletal muscle using single cell RNA-sequencing (scRNA-Seq) and CyTOF revealed roughly 10 major cell types including muscle fibers, endothelial cells, smooth muscle, fibroadipogenic precursors (FAPs), tenocytes, immune cells, satellite cells or MuSCs, and glial cells. The goal was to identify in the interactions between major cell types that participate in skeletal muscle regeneration. To this, it was first sought to define the range of cell types that are in muscle during the regenerative process and capture even transient cell types. we performed scRNA-Seq on mononuclear cells isolated from the hind limb muscles of 8 week old C57/BL6 mice either uninjured, or at days 3, 6, and 10 after an intramuscular (i.m.) notexin injury. Transcriptomic profiles of ˜35,000 single cells were captured and clustering algorithms were used to delineate myogenic, immune, connective tissue, vascular, and glial cell lineages (FIG. 6A), as identified by an unsupervised clustering results for single cell RNA-sequencing of mononuclear cells isolated from a time course of muscle regeneration. Such identification led to the identification of 19 cell types:
Myogenic: MuSCs, activated MuSCs, myoblasts, myocytes, myotubes, and myofibers.
Immune subsets: granulocytes, monocytes, M1 and M2 macrophages, dendritic cells, and lymphoid cells (consisting of B, T, and NK cells).
Connective tissue cell types: fibroadipogenic precursors (FAPs) and fibroblasts.
Vascular cells: smooth muscle and endothelial cells.
Glial cells: Schwann cells.

From the transcriptome profiles of each cell type, a set of markers were identified that optimally delineate these 19 cell types. The marker set uses overlapping and mutually exclusive expression profiles to segregate individual cell types. RNA markers were then translated to a list of 40 previously validated, commercial antibodies that can be used for CODEX (FIG. 6B). To delineate further functional subsets and tissue level changes, we included antibodies against Ki-67 and phosphorylated serine-10 on histone H3 (phospho-H3) to profile cell proliferation, immunoglobulin M (IgM) for necrosis, and types I and III collagen (Collagen I and reticular fibers) found in fibrosis. A unique DNA-barcode was conjugated to each antibody in the skeletal muscle CODEX panel; a 3-step staining process was optimized, and then a standard protocol was created for the detection of these antibodies using CODEX.

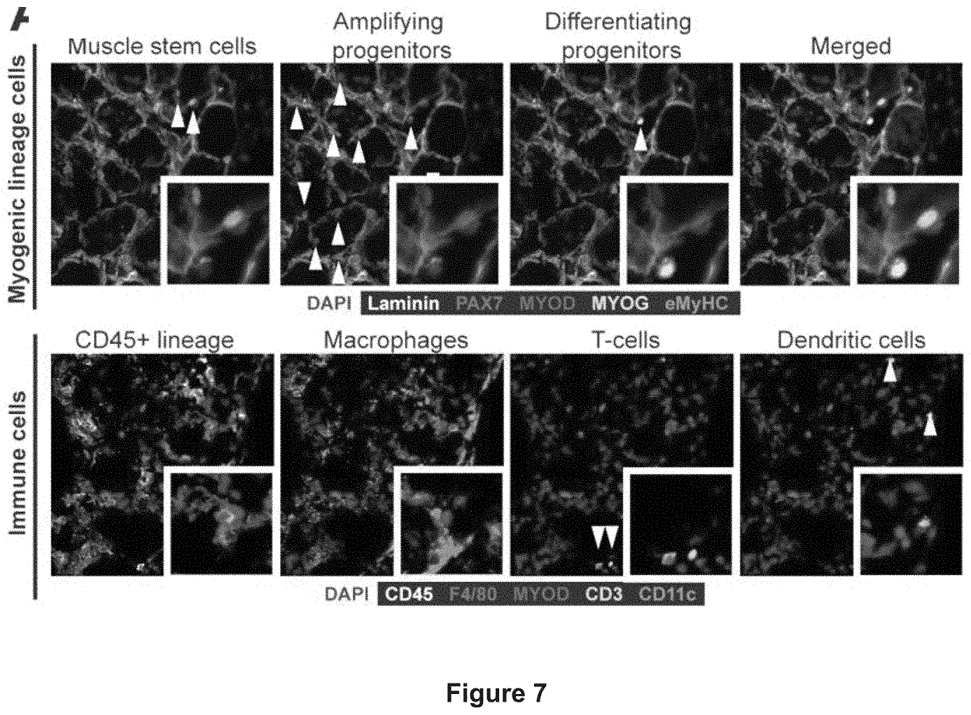

Examples of each cell type, based on expected markers combinations, are clearly identifiable in images generated by the CODEX panel (FIG. 7), which illustrates representative CODEX images of regenerating skeletal muscle showing accurate detection of 40 antibodies and simultaneous visualization of myogenic and immune cell types. These include all 19 aforementioned cell types, along with additional heterogeneity not resolved by scRNA-Seq due to (i) the number of cells analyzed (e.g., few B cells and T cells were detected by scRNA-Seq) or (ii) functional subsets that are not distinct at the transcript level (e.g., differentially localized vascular vs. capillary endothelial cells). Importantly, the tissue architecture and spatial relationships between cell types are preserved. This allows the visualization of interactions between cell types such as Pax7+MuSCs and Myog+differentiating myocytes, or MyoD+myoblasts and F4/80+ macrophages.

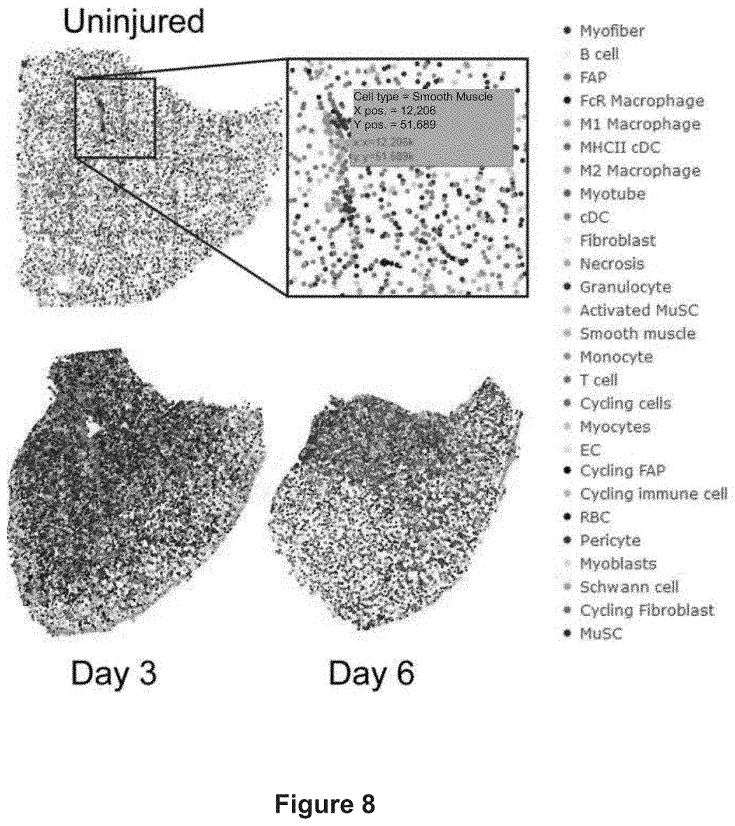

To decipher the cell types in the tissue, cell segmentation was applied and intensities across all 40 antibodies was quantified to classify these cells into distinct cell types using a semi-supervised machine learning algorithm. The algorithm uses an elbow point identification method to (i) identify segmented objects that are likely cells, (ii) remove cells with high autofluorescence, (iii) suppress background in each of the 40 markers to identify cells that have high signal. Cells with high signal are projected in high dimensional space using manifold algorithms such as uniform manifold approximation (UMAP), and clustered using Louvain clustering. Clustering using this subset of cells with high confidence marker expression is more accurate. The labels generated from unsupervised clustering are then used to in machine learning algorithm such as k-nearest neighbor-based label propagation in high dimensional space or training a neural network classifier to label the remaining lower confidence cells. This results in the abundance and spatial dynamics of 27 cell types (>90% of cells are annotated; cell type annotations were manually validated) during regeneration including myogenic cells, immune cells, and fibroblasts (FIG. 8). Specifically, FIG. 8 illustrates representative spatial maps of 27 cell types in uninjured and regenerating TA muscles (days 3 and 6 after injury) of wild type mice. Each dot represents a single cell.

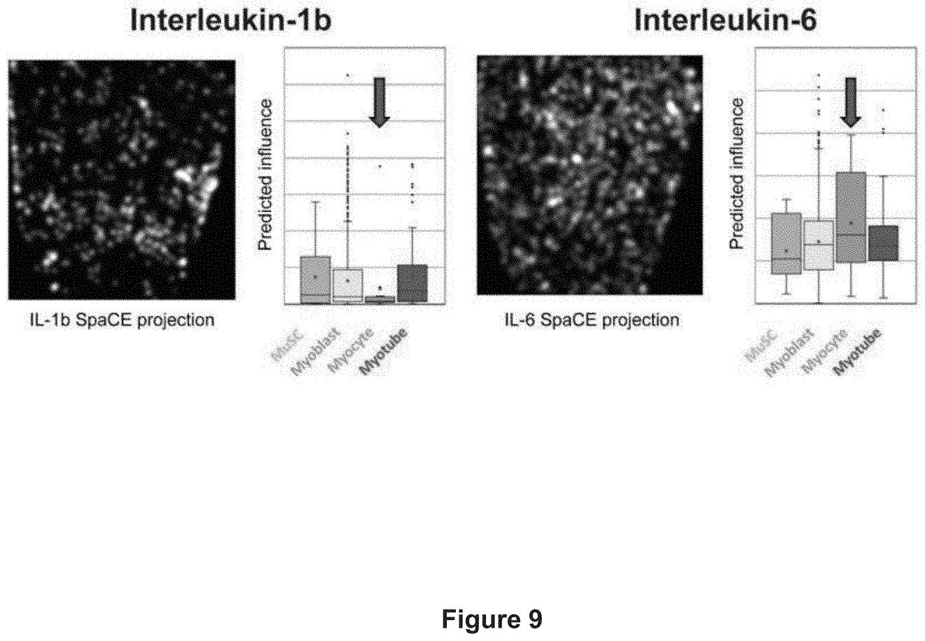

This embodiment generated spatial transcriptome projections of ligands onto regenerating muscle sections (day 3 after a notexin-induced injury; FIG. 9). Moreover, this embodiment used ligands that correlate with the differentiation trajectory of myogenic cells or across cell types to identify signaling molecules that affect cell fate, exemplified by IL-1b and IL-6, which have known disparate effects on myogenic differentiation that were reaffirmed in this embodiment (FIG. 9). Specifically, FIG. 9 illustrates the predictions of interleukins with distinct spatial localization and their quantified “influence” on myogenic subsets. IL-1b and IL-6 are known to inhibit and promote myogenic differentiation, respectively. Arrow indicates that myocytes in regenerating muscle are not near cells secreting IL-1b, but are near sources of IL-6.

Conclusion: This embodiment reveals a wide range of cell types and functional subsets within the tissue while retaining their in vivo spatial positions. This previously unobtainable information will be the basis of measuring cell-cell interactions during tissue regeneration, and used to map single cell transcriptomics data and predict ligand localization. Further, spatial resolution of ligands in relation to cells resolves actual signaling pathways involved in muscle generation.

### Example 2: Modeling of Spatial Accumulation of Tumor Cytokines

Background: Cancer cells secrete immunomodulatory cytokines and angiogenic factors to create a tumorigenic environment. The relative abundance and distribution of cytokines within the tumor microenvironment can promote tumor growth, deflect the immune response, and lead to poor patient outcomes. An array of inflammatory cytokines, including IL-1, IL-6, and tissue necrosis factor alpha (TNF-alpha), have been implicated in promoting colorectal tumor cell proliferation, migration. Therapeutics developed against these cytokines are currently being developed and under clinical trial investigation.

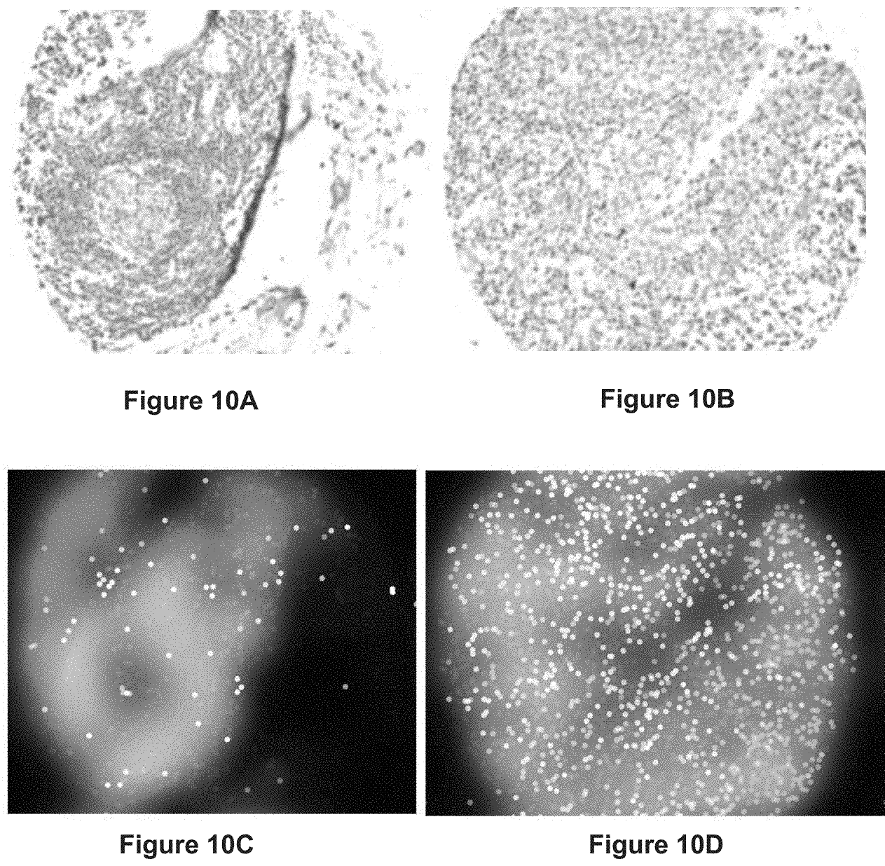

Methods: To obtain spatial data of single cells in colorectal cancer (CRC), CODEX was performed on 35 patient colorectal tumor biopsies. (See e.g., Schürch et al., Coordinated Cellular Neighborhoods Orchestrate Antitumoral Immunity at the Colorectal Cancer Invasive Front. Cell 2020; the disclosure of which is hereby incorporated by reference in its entirety.) Samples represent two distinct types of CRCs, ones with “Crohn's-like reactions” (CLRs) (FIG. 10A) that have longer overall survival, compared to ones with the presence of “diffuse inflammatory infiltrations” (DIIs) (FIG. 10B). Spatial information for each of the 28 cell types was extracted and used to build a spatial transcriptome of each biopsy. Representative bulk RNA seq of cell types found in CRCs including HT-29 colorectal cancer cells (GEO dataset GSE90944) and other cell types were obtained from publicly available data. (See e.g., Ramilowski et al. A draft network of ligand-receptor-mediated multicellular signaling in human. Nature Communications 6, Article number: 7866 (2015); the disclosure of which is hereby incorporated by reference in its entirety.) A dictionary was manually curated to match the annotations of cell types from the CODEX data to each of the bulk RNA-seq datasets. This bulk RNA expression data was superimposed onto the spatial data from CODEX to generate a spatial transcriptome. Relevant genes representing ligands were extracted and diffusion modeling was used to predict the spatial distribution of ligands within the CRC samples.

Results: FIGS. 10C-10D illustrate predicted spatial accumulation of IL6, TNF-alpha, IL1a, and IL1 r1 for a CLR tumor (FIG. 10C) and a DII tumor (FIG. 10D). The CLR tumor (FIG. 10C) shows a defined cytokine localization where the strong prognostic predictor of CRC outcome, interleukin 6 (IL6; visualized in red) is restricted in small regions of the tumor. In contrast, the DII tumor (FIG. 10D), IL6 is diffuse and more abundantly expressed throughout the entire sampled region. This suggests that DII tumors may respond to anti-IL6 antibody treatment (e.g., tocilizumab). Additionally, IL6 signals are mutually exclusive with TNF-alpha in CLRs compared to overlapping accumulation in DIIs.

Conclusion: Such overlapping distributions suggest that therapeutic strategies to target both TNF-alpha and IL-6 could have additive effects in treating DII CRCs.

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method to predict cellular signaling pathways comprising:
obtaining spatial data and expression data, wherein the spatial data identifies cell type and cell position within a sample based on imaging of the tissue, and wherein the expression data identifies genes being expressed in individual cells;
aligning and integrating the spatial and expression data to identify cell linkages between cell type and expression data;
rendering spatial transcriptomics data, wherein the spatial transcriptomics data comprises a transcriptome profile for a cell in the sample in spatial coordinates based on the spatial data and cell linkages;
generating a ligand diffusion map based on spatial data and ligand diffusion information;
measuring effective ligand concentration at cellular positions based on the ligand diffusion map; and
predicting cell-cell signaling in the sample based on the concentration of a ligand at a cellular position within the sample.

2. The method of claim 1, wherein the sample is a tumor from an individual.

3. The method of claim 2, further comprising:
identifying a treatment to block or modulate the cell-cell signaling within the tumor; and
treating an individual with the treatment to block or modulate the cell-cell signaling.

4. The method of claim 1, wherein the expression data includes single cell RNA sequencing data.

5. The method of claim 4, wherein the expression data includes bulk RNA sequencing data.

6. The method of claim 1, wherein the spatial data is obtained from multiparametric tissue imaging.

7. The method of claim 6, wherein the spatial data includes annotated cell type information.

8. The method of claim 7, wherein the spatial data includes single cell protein expression data.

9. The method of claim 7, wherein the expression data includes single cell RNA sequencing (scRNA-Seq) data.

10. The method of claim 9, wherein the aligning and integrating the spatial and expression data comprises:
linking a subset of scRNA-Seq data to annotated cell type to create cell lineages;
linking protein makers to corresponding genes based on the single cell protein expression data and the expression data; and
creating a distance matrix for cells across the scRNA-Seq data and the spatial data.

11. The method of claim 1, wherein generating a ligand diffusion map comprises:
obtaining ligand information from a database, wherein the ligand information includes the ligand diffusion information;
calculating a diffusion kernel for a ligand based on the ligand diffusion information; and
convolving the spatial transcriptomics data with the diffusion kernel.

12. The method of claim 11, wherein the ligand information further comprises at least one of the following: signaling modality, gene name, molecular property, and diffusion constant.

13. The method of claim 1, wherein measuring effective ligand concentration comprises:
convolving the diffusion map with cell radius assess an effective ligand concentration over a cell area; and
outputting ligand concentration for a cell.

14. A non-transitory machine-readable media containing processor instructions, where execution of the instructions causes a processor to predict cellular signaling pathways comprising:
obtaining spatial data and expression data, wherein the spatial data identifies cell type and cell position within a sample based on imaging of the tissue, and wherein the expression data identifies genes being expressed in individual cells;
aligning and integrating the spatial and expression data to identify cell linkages between cell type and expression data;
rendering spatial transcriptomics data, wherein the spatial transcriptomics data comprises a transcriptome profile for a cell in the sample in spatial coordinates based on the spatial data and cell linkages;
generating a ligand diffusion map based on spatial data and ligand diffusion information;
measuring effective ligand concentration at cellular positions based on the ligand diffusion map; and
predicting cell-cell signaling in the sample based on the concentration of a ligand at a cellular position within the sample.

15. The non-transitory machine-readable media of claim 14, wherein the sample is a tumor from an individual.

16. The non-transitory machine-readable media of claim 15, wherein the instructions further comprise identifying a treatment to block or modulate the cell-cell signaling within the tumor.

17. The non-transitory machine-readable media of claim 14, wherein the expression data includes single cell RNA sequencing data.

18. The non-transitory machine-readable media of claim 17, wherein the expression data includes bulk RNA sequencing data.

19. The non-transitory machine-readable media of claim 14, wherein the spatial data is obtained from multiparametric tissue imaging.

20. The non-transitory machine-readable media of claim 19, wherein the spatial data includes annotated cell type information.

21. The non-transitory machine-readable media of claim 20, wherein the spatial data includes single cell protein expression data.

22. The non-transitory machine-readable media of claim 20, wherein the expression data includes single cell RNA sequencing (scRNA-Seq) data.

23. The non-transitory machine-readable media of claim 22, wherein the aligning and integrating the spatial and expression data comprises:
linking subset of scRNA-Seq data to annotated cell type to create cell lineages;
linking protein makers to corresponding genes based on the single cell protein expression data and the expression data; and
creating a distance matrix for cells across the scRNA-Seq data and the spatial data.

24. The non-transitory machine-readable media of claim 14, wherein generating a ligand diffusion map comprises:
obtaining ligand information from a database, wherein the ligand information includes the ligand diffusion information;
calculating a diffusion kernel for a ligand based on the ligand diffusion information; and
convolving the spatial transcriptomics data with the diffusion kernel.

25. The non-transitory machine-readable media of claim 24, wherein the ligand information further comprises at least one of the following: signaling modality, gene name, molecular property, and diffusion constant.

26. The non-transitory machine-readable media of claim 14, wherein measuring effective ligand concentration comprises:
convolving the diffusion map with cell radius assess an effective ligand concentration over a cell area; and
outputting ligand concentration for a cell.

27. A system for predicting cellular signaling pathways comprising:
a processor; and
a memory readable by the processor, wherein the memory contains instructions that when read by the processor direct the processor to:
obtain spatial data and expression data, wherein the spatial data identifies cell type and cell position within a sample based on imaging of the tissue, and wherein the expression data identifies genes being expressed in individual cells;
align and integrating the spatial and expression data to identify cell linkages between cell type and expression data;
render spatial transcriptomics data, wherein the spatial transcriptomics data comprises a transcriptome profile for a cell in the sample in spatial coordinates based on the spatial data and cell linkages;
generate a ligand diffusion map based on spatial data and ligand diffusion information;
measure effective ligand concentration at cellular positions based on the ligand diffusion map; and
predict cell-cell signaling in the sample based on the concentration of a ligand at a cellular position within the sample.
